(19) United States
(12) Patent Application Publication
(10) Pub. No.: US 2015/0259730 A1
(43) Pub. Date: Sep. 17, 2015

(54) MICROFLUIDIC CHIP, OPERATING SYSTEM AND OPERATING METHOD OF THE MICROFLUIDIC CHIP FOR FLUORESCENCE IN SITU HYBRIDIZATION

(71) Applicant: National Tsing Hua University, Hsinchu City (TW)

(72) Inventors: Gwo-Bin Lee, Hsinchu City (TW); Ta-Sen Yeh, Hsinchu City (TW); Tse-Ching Chen, Hsinchu City (TW); Chien-Hsuan Tai, Hsinchu City (TW); Kai-Jie Kao, Hsinchu City (TW)

(21) Appl. No.: 14/483,735

(22) Filed: Sep. 11, 2014

(30) Foreign Application Priority Data
Mar. 14, 2014 (TW) 103109380

(51) Int. Cl. C12Q 1/68; B01L 3/00

(52) U.S. Cl. CPC C12Q 1/6825; B01L 3/5027; B01L 3/508; B01L 3/567; B01L 2300/18

(57) ABSTRACT

A microfluidic chip is provided, including: a plurality of first storage tanks and a plurality of second storage tanks that respectively store different solutions for a preprocess and hybridization process of the fluorescence in situ hybridization; a transmission unit adjacent to the first storage tanks; a reaction unit adjacent to the second storage tanks for a biological target to be placed thereon; a plurality of first valves disposed between the first storage tanks and the transmission unit; and a plurality of second valves disposed between the second storage tanks and the reaction unit, wherein the first valves and the second valves are opened in a predetermined sequence so as to transmit the solutions of the first storage tanks and the second storage tanks to the reaction unit, such that the solutions perform the preprocess and hybridization process of the fluorescence in situ hybridization to the biological target in sequence.

### BACKGROUND OF THE INVENTION

1. Field of the Invention

The present invention relates to microfluidic chips and operating methods thereof, and, more particularly, to a microfluidic chip, an operating system and an operating method of the microfluidic chip for fluorescence in situ hybridization.

2. Description of Related Art

Health is always a concern, especially in recent years, for population structure is aged, living quality is improved, medical quality is promoted, and biological technology industry has been one of the most popular industries of nowadays. The biological technology can be widely applied to, for example, fields of medicine, medical care, agriculture, food, environmental resource, and other chemical industries. One of the most important fields is a quick diagnosis technique of cancer, and how to prevent or effectively diagnose cancer is a well-discussed topic for an early observation and early treatment.

According to the research, HER1 and HER2 (human epidermal growth factor receptor 1 and 2) genes have over-expression in several types of cancers. Therefore, HER1 and HER2 genes are variously utilized as indicators for clinic cancer prediction. For example, it is discovered that 5 to 10 percent of gastric cancer are induced by the over-expression of HER2 gene, and the patient can be treated by using the targeted drug, Herceptin at an early stage, so as to extend the cancer survival rate.

Currently, a normal method to detect the over-expression of HER2 gene is fluorescence in situ hybridization (FISH) technique, which uses fluorescent probes having a small segment of DNA sequences to precisely match and combine with certain DNA sequences, and the occurrence of over-expression of HER2 gene is determined by finding fluorescent spots via a fluorescence microscopy. However, the reagent for the FISH technique is too expensive and the procedure is very complicated, such that error may occur and contamination may be caused due to an inappropriate human operation.

Therefore, how to overcome the problems in the prior art is an issue desired to be solved.

### SUMMARY OF THE INVENTION

According to the above drawbacks of the prior art, the present invention provides a microfluidic chip for fluorescence in situ hybridization, comprising: a plurality of first storage tanks and a plurality of second storage tanks that respectively store different solutions for a preprocess and hybridization process of the fluorescence in situ hybridization; a transmission unit adjacent to the first storage tanks; a reaction unit adjacent to the second storage tanks for a biological target to be place thereon; a plurality of first valves disposed between the first storage tanks and the transmission unit; and a plurality of second valves disposed between the second storage tanks and the reaction unit, wherein the first valves and the second valves are opened in a predetermined sequence so as to transmit the solutions of the first storage tanks and the second storage tanks to the reaction unit, such that the solutions perform the preprocess and hybridization process of the fluorescence in situ hybridization to the biological target in sequence.

The present invention also provides an operating system for fluorescence in situ hybridization, comprising: an operating platform; and a microfluidic chip disposed on the operating platform and comprising: a plurality of first storage tanks and a plurality of second storage tanks that respectively store different solutions for a preprocess and hybridization process of the fluorescence in situ hybridization; a transmission unit adjacent to the first storage tanks; a reaction unit adjacent to the second storage tanks for a biological target to be placed thereon; a plurality of first valves disposed between the first storage tanks and the transmission unit; and a plurality of second valves disposed between the second storage tanks and the reaction unit, wherein the first valves and the second valves are opened in a predetermined sequence so as to transmit the solutions of the first storage tanks and the second storage tanks to the reaction unit, such that the solutions perform the preprocess and hybridization process of the fluorescence in situ hybridization to the biological target in sequence.

The present invention further provides an operating method of a microfluidic chip for fluorescence in situ hybridization, comprising: providing a microfluidic chip having a plurality of first storage tanks, a plurality of second storage tanks, a transmission unit, a reaction unit, a plurality of first valves and a plurality of second valves, wherein the first valves are disposed between the first storage tanks and the transmission unit, the second valves are disposed between the second storage tanks and the reaction unit, and a biological target is disposed on the reaction unit; storing different solutions for a preprocess and hybridization process of the fluorescence in situ hybridization to the first storage tanks and the second storage tanks, respectively; and opening the first valves and the second valves in a predetermined sequence so as to transmit the solutions of the first storage tanks and the second storage tanks to the reaction unit, such that the solutions perform the preprocess and hybridization process of the fluorescence in situ hybridization to the biological target in sequence.

It can be seen that the present invention relates to a microfluidic chip, an operating system and an operating method of the microfluidic chip for fluorescence in situ hybridization. The microfluidic chip includes storage tanks, valves, a transmission unit, and a reaction unit, and solutions stored in the storage tanks can be successively transmitted to the reaction unit in accordance to a preprocess and hybridization process of the fluorescence in situ hybridization, such that the preprocess and hybridization process are performed to a biological target in sequence through the solutions.

Therefore, the present invention achieves the preprocess and hybridization process of the fluorescence in situ hybridization in an automation manner, and the microfluidic chip brings various efficacy such as being cost-effective, compact, disposable, user-friendly and widely applicable. Given the above efficacy, the microfluidic chip may quickly diagnose cancers and relative diseases with the fluorescence in situ hybridization technique, and will not generate error or cause contamination due to an inappropriate human operation.

### BRIEF DESCRIPTION OF DRAWINGS

FIG. 1 is a top view of a microfluidic chip for fluorescence in situ hybridization according to the present invention;

FIG. 2 is a structural scheme view of an operating system for fluorescence in situ hybridization according to the present invention;

FIG. 3 is a flow chart of an operating method of a microfluidic chip for fluorescence in situ hybridization according to the present invention; and

FIGS. 4A and 4B are actual images in a diagnosis of breast cancer cells utilizing a microfluidic chip for fluorescence in situ hybridization according to the present invention.

### DETAILED DESCRIPTION OF THE INVENTION

The following illustrative embodiments are provided to illustrate the disclosure of the present invention, these and other advantages and effects can be apparently understood by those in the art after reading the disclosure of this specification.

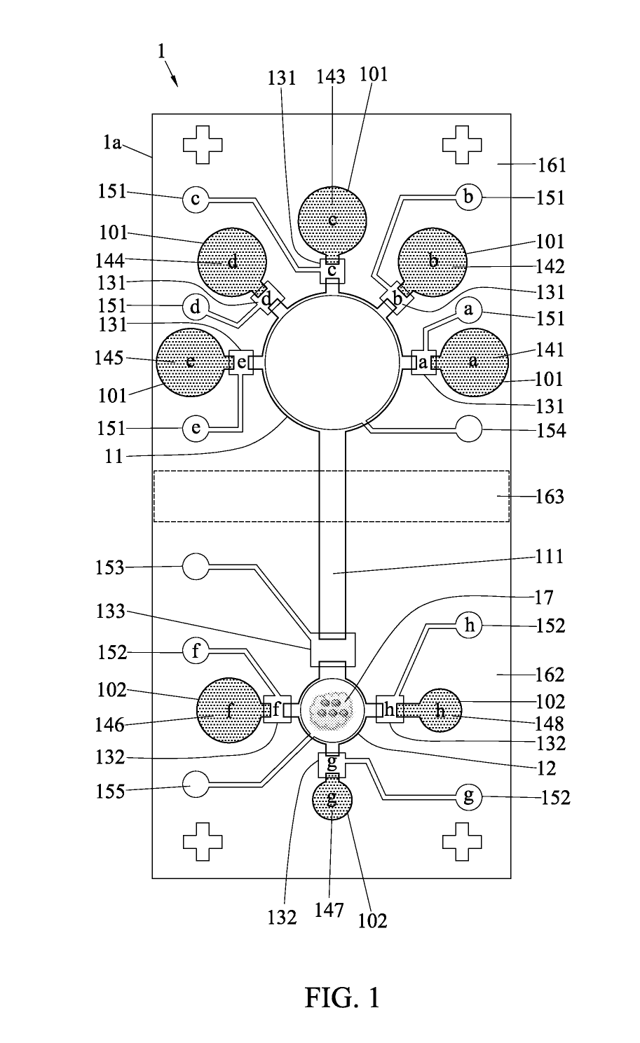

FIG. 1 is a top view of a microfluidic chip 1 for fluorescence in situ hybridization according to the present invention. The microfluidic chip 1 comprises a plurality of first storage tanks 101, a plurality of second storage tanks 102, a transmission unit 11, a reaction unit 12, a plurality of first valves 131 and a plurality of second valves 132.

The first storage tanks 101 and the second storage tanks 102 respectively store different solutions for a preprocess and hybridization process of the fluorescence in situ hybridization.

In an embodiment, the solutions stored in the first storage tanks 101 include NaSCN solution 141, pepsin solution 142, water solution 143, alcohol solution 144 and fluorescent dye solution 145, and the solutions stored in the second storage tanks 102 include cleaning solution 146, probe solution 147 and waste 148. Also, the fluorescent dye solution 145 may be a DAPI (4′,6-diamidino-2-phenylindole) dye.

The NaSCN solution 141, pepsin solution 142, water solution 143 and alcohol solution 144 are utilized to perform the preprocess of the fluorescence in situ hybridization to the biological target 17.

The transmission unit 11 is adjacent to the first storage tanks 101, and the reaction unit 12 is adjacent to the second storage tanks 102 and placed with a biological target 17.

The plurality of first valves 131 are disposed between the first storage tanks 101 and the transmission unit 11, and the plurality of second valves 132 are disposed between the second storage tanks 102 and the reaction unit 12.

In the microfluidic chip 1, the first valves 131 and the second valves 132 are respectively opened in accordance to a sequence of the preprocess and hybridization process of the fluorescence in situ hybridization, so as to successively transmit the solutions of the first storage tanks 101 and the second storage tanks 102 to the reaction unit 12, such that the solutions perform the preprocess and hybridization process of the fluorescence in situ hybridization to the biological target 17 in sequence.

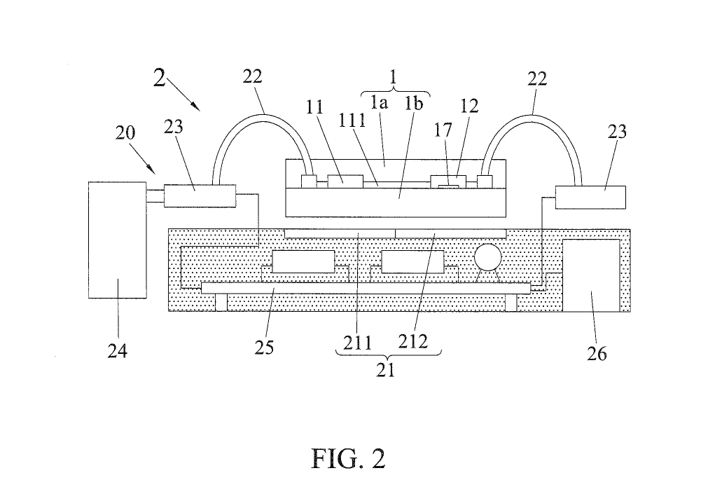

The microfluidic chip 1 comprises an upper plate 1a and a substrate 1b, as shown in FIG. 2, and the upper plate 1a can be fabricated my a PDMS material.

The microfluidic chip 1 comprises a cooling region 161, a heating region 162, and an isolating region 163 between the cooling region 161 and the heating region 162. The first storage tanks 101 and the transmission unit 11 are in the cooling region 161, the second storage tanks 102 and the reaction unit 12 are in the heating region 162, and the heating region 162 has a temperature (such as 75° C.) higher than a temperature (such as 37° C.) of the cooling region 161.

The transmission unit 11 has a connection portion 111 extending from the cooling region 161 through the isolating region 163 to the heating region 162 and adjacent to the reaction unit 12.

The microfluidic chip 1 comprises a third valve 133, a plurality of first pores 151, a plurality of second pores 152, a third pore 153, a fourth pore 154, and a fifth pore 155. The third valve 133 is disposed between the connection portion 111 and the reaction unit 12. The first pores 151, the second pores 152 and the third pore 153 are connected to the first valves 131, the second valves 132 and the third valve 133, respectively. The fourth pore 154 and the fifth pore 155 are connected to the transmission unit 11 and the reaction unit 12, respectively.

FIG. 2 is a structural scheme view of an operating system 2 for fluorescence in situ hybridization according to the present invention. Please also refer to FIG. 1. The operating system 2 comprises an operating platform 20 and a microfluidic chip 1 disposed on the operating platform 20.

The microfluidic chip 1 comprises a plurality of first storage tanks 101, a plurality of second storage tanks 102, a transmission unit 11, a reaction unit 12, a plurality of first valves 131 and a plurality of second valves 132.

The first storage tanks 101 and the second storage tanks 102 respectively store different solutions for a preprocess and hybridization process of the fluorescence in situ hybridization. The transmission unit 11 is adjacent to the first storage tanks 101, and the reaction unit 12 is adjacent to the second storage tanks 102 and placed with a biological target 17. The plurality of first valves 131 are disposed between the first storage tanks 101 and the transmission unit 11, and the plurality of second valves 132 are disposed between the second storage tanks 102 and the reaction unit 12.

In the microfluidic chip 1, the first valves 131 and the second valves 132 are respectively opened in accordance to a sequence of the preprocess and hybridization process of the fluorescence in situ hybridization, so as to successively transmit the solutions of the first storage tanks 101 and the second storage tanks 102 to the reaction unit 12, such that the solutions perform the preprocess and hybridization process of the fluorescence in situ hybridization to the biological target 17 in sequence.

The microfluidic chip 1 comprises a cooling region 161, a heating region 162, and an isolating region 163 between the cooling region 161 and the heating region 162. The first storage tanks 101 and the transmission unit 11 are in the cooling region 161, and the second storage tanks 102 and the reaction unit 12 are in the heating region 162. The heating region 162 has a temperature higher than a temperature of the cooling region 161, such that the solutions in the first storage tanks 101 and the second storage tanks 102 are at suitable temperatures.

In an embodiment, the solutions stored in the first storage tanks 101 are NaSCN solution 141, pepsin solution 142, water solution 143, alcohol solution 144 and fluorescent dye solution 145 ,respectively. The solutions stored in the second storage tanks 102 are cleaning solution 146, probe solution 147 and waste 148, respectively.

The microfluidic chip 1 comprises a plurality of first pores 151 and a plurality of second pores 152 connected with the first valves 131 and the second valves 132, respectively.

In addition, other technical contents regarding the microfluidic can be referred from the illustration of FIG. 1, and are thus omitted.

The operating platform 20 comprises a temperature control device 21 having a first heating unit 211 corresponding to the cooling region 161 and a second heating unit 212 corresponding to the heating region 162.

In an embodiment, the operating platform 20 comprises a plurality of connected air pipes 22, at least one electromagnetic valve 23, an air pressure generating device 24 such as a steady pressure air compressor. The air pipes 22 are connected with the first pores 151, the second pores 152, the third pore 153, the fourth pore 154 and the fifth pore 155, respectively, and the electromagnetic valve 23 controls the air pressure generating device 24 to generate negative pressure to the first pores 151, the second pores 152, the third pore 153, the fourth pore 154 and the fifth pore 155.

In an embodiment, the control platform 20 comprises a control circuit 25 or a power supply 26. The control circuit 25 controls the electromagnetic valve 23, and the power supply 26 provides power to the control circuit 25 and/or the temperature control device 21.

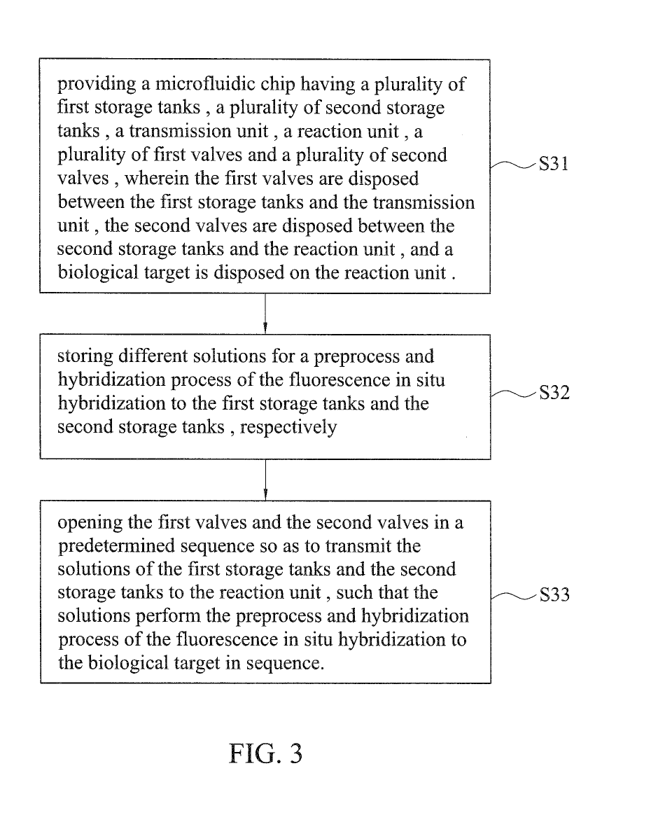

FIG. 3 is a flow chart of an operating method of a microfluidic chip for fluorescence in situ hybridization according to the present invention. Please also refers to FIG. 1.

Step S31 shows providing a microfluidic chip 1 having a plurality of first storage tanks 101, a plurality of second storage tanks 102, a transmission unit 1, a reaction unit 12, a plurality of first valves 131 and a plurality of second valves 132. The first valves 131 are disposed between the first storage tanks 101 and the transmission unit 11, the second valves 132 are disposed between the second storage tanks 102 and the reaction unit 12, and a biological target 17 is disposed on the reaction unit 12.

Step S32 shows storing different solutions for a preprocess and hybridization process of the fluorescence in situ hybridization to the first storage tanks 101 and the second storage tanks 102, respectively, wherein the solutions stored in the first storage tanks 101 include NaSCN solution 141, pepsin solution 142, water solution 143, alcohol solution 144 and fluorescent dye solution 145, and the solutions stored in the second storage tanks 102 include cleaning solution 146, probe solution 147 and waste 148.

Step S33 shows opening the first valves 131 and the second valves 132 in a predetermined sequence so as to transmit the solutions of the first storage tanks 101 and the second storage tanks 102 to the reaction unit 12, such that the solutions perform the preprocess and hybridization process of the fluorescence in situ hybridization to the biological target 17 in sequence.

The preprocess of the fluorescence in situ hybridization comprises: (1) using the NaSCN solution 141 (for example, at 73° C.) to soak the biological target 17, and using the water solution 143 to perform a first cleaning to the biological target 17; (2) using the pepsin solution 142 (for example, of 0.005%) to decompose membrane protein of the biological target 17, and using the water solution 143 to perform a second cleaning to the biological target 17; (3) using the alcohol solution 144 (sequentially of 70%, 85% and 100%) to dehydrate the biological target 17.

The hybridization process of the fluorescence in situ hybridization comprises: (1) using probes of the probe solution 147 to perform a hybridization to gene of the biological target 17, wherein the probe solution 147 has orange fluorescent probes of HER2 gene and green fluorescent probes of CEP-17 (chromosome number 17); (2) using the cleaning solution 146 to wash away the probes that did not perform the hybridization to gene of the biological target 17; (3) using the fluorescent dye solution (such as DAPI dye) 145 to dye nuclei of the biological target 17.

After the preprocess and hybridization process of the fluorescence in situ hybridization are completed, the biological target 17 can be placed at a fluorescence microscopy (not illustrated), and find fluorescent spots of cells of the biological target 17 via the fluorescence microscopy so as to determine and confirm whether an over-expression of HER2 gene occurs in the cells.

In addition, an operating method of components such as storage tanks, solutions, valves and pores of the microfluidic chip 1 is described as follows.

(1) The pressure generating device 24 is controlled by using the electromagnetic valve 23 shown in FIG. 2 to generate negative or positive pressure, and the negative pressure is applied to the first pore 151a of FIG. 1 connected with the air pipe 22, such that the NaSCN solution 141 of the first storage tanks 101a is transmitted to the transmission unit 11. The negative pressure is applied to the third pore 153 to open the third valve 133, and the positive pressure is applied to the fourth pore 154 to transmit the NaSCN solution 141 in the transmission unit 11 to the reaction unit 12 via the connection portion 111, such that the NaSCN solution 141 soaks the biological target 17 and generate the waste 148. The negative pressure is applied to the second pore 152h to open the second valve 132h, and the positive pressure is applied to the fifth pore 155 to transmit the waste 148 in the reaction unit 12 to the second storage tank 102h or the outside thereof.

(2) Similar to the operating method of (1), the water solution 143 of the first storage tanks 101c is transmitted to the reaction unit 12 via the first valve 131c (by applying the negative pressure to the first pore 151c), the transmission unit 11, the connection portion 111 and the third valve 133 in sequence. The water solution 143 performs a first cleaning to the biological target 17, and transmits the waste 148 to the second storage tank 102h or the outside thereof.

(3) Also, the pepsin solution 142 of the first storage tanks 101b is transmitted to the reaction unit 12 via the first valve 131b (by applying the negative pressure to the first pore 151b), the transmission unit 11, the connection portion 111 and the third valve 133 in sequence. The pepsin solution 142 decompose membrane protein of the biological target 17, and then the water solution 143 performs a second cleaning to the biological target 17 and transmits the waste 148 to the second storage tank 102h or the outside thereof.

(4) The alcohol solution 144 of the first storage tank 101d is transmitted to the reaction unit 12 via the first valve 131d (by applying the negative pressure to the first pore 151d), the transmission unit 11, the connection portion 111 and the third valve 133 in sequence. The alcohol solution 144 dehydrates the biological target 17 and transmits the waste 148 to the second storage tank 102h or the outside thereof.

(5) The probe solution 147 of the first storage tank 102g is transmitted to the reaction unit 12 via the second valve 132g (by applying the negative pressure to the second pore 152g). The probe solution 147 is utilized to perform a hybridization to gene of the biological target 17.

(6) The cleaning solution 146 of the second storage tank 102f is transmitted to the reaction unit 12 via the second valve 132f (by applying the negative pressure to the second pore 152f). The cleaning solution 146 is utilized to wash away the probes that did not perform the hybridization to gene of the biological target 17, and the waste 148 is transmitted to the second storage tank 102h or the outside thereof.

(7) The fluorescent dye solution 145 is transmitted to the reaction unit 12 via the first valve 131e (by applying the negative pressure to the first pore 151e), the transmission unit 11, the connection portion 111 and the third valve 133 in sequence. The fluorescent dye solution 145 is utilized to dye nuclei of the biological target 17.

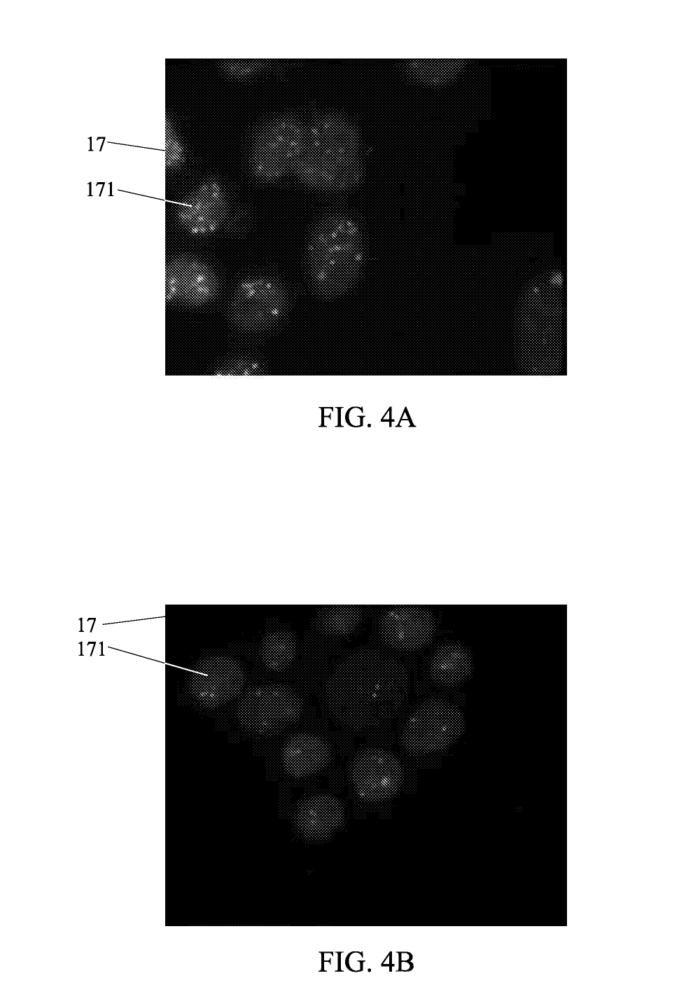

FIGS. 4A and 4B are actual images in a diagnosis of breast cancer cells utilizing a microfluidic chip for fluorescence in situ hybridization according to the present invention.

As illustrated in FIGS. 4A and 4B, the orange spots represent the HER2 gene, and the green spots represent CEP-17. The over-expression of HER2 gene is defined by a ratio of the amount of orange spots in 60 cells to the amount of green spots, and the result is determined positive when the ratio is greater than 2.2, otherwise is determined negative. However, when there are more than six green spots in a single cell, i.e., more than six CEP-17, it is called polysomy, which is also a pattern of the over-expression.

In FIG. 4A, the target 17 presents polysomy and a positive result, that means breast cancer cells of SKBR-3 are detected in this embodiment. In FIG. 4B, the ratio of HER2 to CEP-17 of the biological target 17 is 1.067 and presents a negative result, that means breast cancer cells of MDA-MB-468 are detected in this embodiment.

It can be seen that the present invention relates to a microfluidic chip, operating system and operating method of the microfluidic chip for fluorescence in situ hybridization. The microfluidic chip mainly includes elements such as storage tanks, valves, transmission unit, reaction unit disposed therein, and solutions stored in the storage tanks can be successively transmitted to the reaction unit in accordance to a preprocess and hybridization process of the fluorescence in situ hybridization, such that the preprocess and hybridization process are performed to a biological target in sequence through the solutions.

Therefore, the present invention achieves the preprocess and hybridization process of the fluorescence in situ hybridization in an automation manner, and the microfluidic chip brings various efficacy such as being cost-effective, compact, disposable, user-friendly and widely applicable. Given the above efficacy, the microfluidic chip may quickly diagnose cancers and relative diseases with the fluorescence in situ hybridization technique, and will not generate error or cause contamination due to an inappropriate human operation.

The above embodiments only exemplarily specify the concept and effect of the invention, but not intend to limit the invention. Any person skilled in the art can perform modifications and adjustments on the above embodiments without departing the spirit and category of the invention.

What is claimed is:

1. A microfluidic chip for fluorescence in situ hybridization, comprising:
a plurality of first storage tanks and a plurality of second storage tanks that respectively store different solutions for a preprocess and hybridization process of the fluorescence in situ hybridization;
a transmission unit adjacent to the first storage tanks;
a reaction unit adjacent to the second storage tanks for a biological target to be placed thereon;
a plurality of first valves disposed between the first storage tanks and the transmission unit; and
a plurality of second valves disposed between the second storage tanks and the reaction unit,
wherein the first valves and the second valves are opened in a predetermined sequence so as to transmit the solutions of the first storage tanks and the second storage tanks to the reaction unit, such that the solutions perform the preprocess and hybridization process of the fluorescence in situ hybridization to the biological target in sequence.

2. The microfluidic chip of claim 1, wherein the solutions stored in the first storage tanks include NaSCN solution, pepsin solution, water solution, alcohol solution and fluorescent dye solution, and the solutions stored in the second storage tanks include cleaning solution, probe solution and waste.

3. The microfluidic chip of claim 2, wherein the NaSCN solution, pepsin solution, water solution, alcohol solution and fluorescent dye solution are utilized to perform the preprocess of the fluorescence in situ hybridization to the biological target, and the fluorescent dye, cleaning, probe solutions are utilized to perform the hybridization process of the fluorescence in situ hybridization to the biological target.

4. The microfluidic chip of claim 1, further comprising a cooling region, a heating region, and an isolating region therebetween, wherein the first storage tanks are in the cooling region, the second storage tanks and the reaction unit are in the heating region, and the heating region has a temperature higher than that of the cooling region.

5. The microfluidic chip of claim 1, further comprising a plurality of first pores connected with the first valves and a plurality of second pores connected with the second valves.

6. The microfluidic chip of claim 1, further comprising a third valve and a third pore connected with the third valve, wherein the transmission unit has a connection portion, and the third valve is disposed between the connection portion and the reaction unit.

7. An operating system for fluorescence in situ hybridization, comprising:
an operating platform; and
a microfluidic chip disposed on the operating platform and comprising:
a plurality of first storage tanks and a plurality of second storage tanks that respectively store different solutions for a preprocess and hybridization process of the fluorescence in situ hybridization;
a transmission unit adjacent to the first storage tanks;
a reaction unit adjacent to the second storage tanks for a biological target to be placed thereon;
a plurality of first valves disposed between the first storage tanks and the transmission unit; and
a plurality of second valves disposed between the second storage tanks and the reaction unit,
wherein the first valves and the second valves are opened in a predetermined sequence so as to transmit the solutions of the first storage tanks and the second storage tanks to the reaction unit, such that the solutions perform the preprocess and hybridization process of the fluorescence in situ hybridization to the biological target in sequence.

8. The operating system of claim 7, wherein the microfluidic chip has a cooling region, a heating region, and an isolating region therebetween, the first storage tanks are in the cooling region, the second storage tanks and the reaction unit are in the heating region, and the heating region has a temperature higher than that of the cooling region.

9. The operating system of claim 8, wherein the operating platform comprises a temperature control device having a first heating unit corresponding to the cooling region and a second heating unit corresponding to the heating region.

10. The operating system of claim 7, wherein the microfluidic chip further comprises a plurality of first pores connected with the first valves and a plurality of second pores connected with the second valves.

11. The operating system of claim 10, wherein the operating platform further comprises a plurality of connected air pipes, at least one electromagnetic valve, and an air pressure generating device, the air pipes are connected with the first pores and the second pores, and the electromagnetic valve controls the air pressure generating device to generate negative pressure to the first pores or positive pressure to the second pores.

12. The operating system of claim 11, wherein the operating platform further comprises a control circuit for controlling the electromagnetic valve.

13. An operating method of a microfluidic chip for fluorescence in situ hybridization, comprising:
providing a microfluidic chip having a plurality of first storage tanks, a plurality of second storage tanks, a transmission unit, a reaction unit, a plurality of first valves and a plurality of second valves, wherein the first valves are disposed between the first storage tanks and the transmission unit, the second valves are disposed between the second storage tanks and the reaction unit, and a biological target is disposed on the reaction unit;
storing different solutions for a preprocess and hybridization process of the fluorescence in situ hybridization in the first storage tanks and the second storage tanks, respectively; and
opening the first valves and the second valves in a predetermined sequence so as to transmit the solutions of the first storage tanks and the second storage tanks to the reaction unit, such that the solutions perform the preprocess and hybridization process of the fluorescence in situ hybridization to the biological target in sequence.

14. The operating method of claim 13, wherein the solutions stored in the first storage tanks include NaSCN solution, pepsin solution, water solution, alcohol solution and fluorescent dye solution, and the solutions stored in the second storage tanks include cleaning solution, probe solution and waste.

15. The operating method of claim 14, wherein the preprocess of the fluorescence in situ hybridization comprises:
using the NaSCN solution to soak the biological target, and using the water solution to perform a first cleaning to the biological target;
using the pepsin solution to decompose membrane protein of the biological target, and using the water solution to perform a second cleaning to the biological target; and
using the alcohol solution to dehydrate the biological target.

16. The operating method of claim 14, wherein the hybridization process of the fluorescence in situ hybridization comprises:
using probes of the probe solution to perform a hybridization to gene of the biological target;
using the cleaning solution to wash away the probes that did not perform the hybridization to gene of the biological target;
using the fluorescent dye solution to dye the biological target.
